(19) United States
(12) Patent Application Publication
(10) Pub. No.: US 2010/0280363 A1
(43) Pub. Date: Nov. 4, 2010

(54) Electromagnetic Navigation of Medical Instruments for Cardiothoracic Surgery

(75) Inventors: James Skarda, Lake Elmo, MN (US); Mark Stewart, Lino Lakes, MN (US); James Keogh, Maplewood, MN (US); David Francischelli, Anoka, MN (US); Kenneth C. Gardeski, Plymouth, MN (US); Thomas A. Poss, St. Louis Park, MN (US); Michael Neidert, Salthill (IE)

Correspondence Address: Medtronic CardioVascular, Mounds View Facility South, 8200 Coral Sea Street N.E., Mounds View, MN, 55112, US

(73) Assignee: Medtronic, Inc., Minneapolis, MN (US)

(21) Appl. No.: 12/767,163

(22) Filed: Apr. 26, 2010

Related U.S. Application Data
(60) Provisional application No. 61/214,591, filed on Apr. 24, 2009.

(51) Int. Cl. A61B 5/05

(52) U.S. Cl. 600/424

(57) ABSTRACT

The present invention provides devices, instruments, systems, and methods to navigate medical instruments within the thoracic cavity. More specifically, the present invention provides a navigation system comprising medical instruments having electromagnetic tracking functionality and the integration of previously acquired imaging into a user interface of the navigation system.

### CROSS-REFERENCE TO RELATED APPLICATION

This application claims the benefit under 35 USC §119(e) of U.S. Provisional Patent Application No. 61/214,591, filed Apr. 24, 2009, which is incorporated herein by reference in its entirety and for all purposes.

### TECHNICAL FIELD

The present invention relates to electromagnetically monitoring, guiding, tracking, or otherwise navigating one or more medical instrument within a predetermined portion of the anatomy of a patient during a medical procedure. More particularly, the present invention relates to electromagnetically monitoring, guiding, tracking, or otherwise navigating one or more medical instrument within a predetermined portion of the anatomy of a patient during minimally invasive cardiothoracic surgery.

### BACKGROUND

A known method of guidance for medical instruments used in minimally invasive and robotic cardiothoracic surgery is of endoscopic visualization. Images from endoscopic light guides and cameras within the thoracic cavity are displayed on a video monitor that is viewed by the surgeon. The effective use of this method depends on there being sufficient open space within the working area of the body. Various retractors and tissue spreading instruments are required to hold tissues away from the working field within the body. Pressurized gasses are introduced to the thoracic cavity to help create space in which to work with a sufficient field of view. In addition, a lung may be deflated to drop the lung away from the working field. Without sufficient space and field of view, it can be difficult for a surgeon to recognize the anatomical location and identity of structures viewed on the video display. This requirement for space surrounding the working field has the effect of limiting the regions that can be safely and confidently accessed by minimally invasive techniques. For example, it can be very difficult for a surgeon to endoscopically visualize the passage of instruments through the spaces posterior to and around portions of the heart such as the transverse and oblique sinuses. Due to these limitations, some procedures are not attempted using minimally invasive techniques.

Other fields of medical practice have adopted new methods of tracking and guiding catheters and medical instruments within certain regions of interest within the body. The medical fields using such technologies include cardiac electrophysiology, neurosurgery, and spinal surgery. Various means of navigation for catheters and neurological medical instruments have been devised. These allow the tracking of such catheters or instruments within one or more of the cardiovascular system, cranial, and spinal regions of the body.

Electrophysiologists use systems that guide the navigation and placement of catheters within the cardiovascular system. Neurosurgeons use such systems to guide medical instruments within body spaces such as the cranium and spinal region. Spinal surgeons use such systems to guide precise placement and attachment of repair structures in the spine. These current navigation technologies include electromagnetic, electric field, and ultrasound based methods.

A system referred to as LocaLisa uses electric field based localization and navigation. The LocaLisa system uses three pairs of electrical patches connected to the body. One set is oriented in each of the three axes, side-to-side, front-to-back, and head-to-toe (patches on neck and thigh). A 40.1 KHz, 40.2 KHz, and 40.3 KHz signal is transmitted between each of the three sets of patches, respectively. Electrodes on devices within the cardiovascular system pick up these three signals. Any electrode in contact with the vascular system or electrically conductive tissue that can be monitored outside of the body to pick up the three signals can then be tracked in three-dimensional space. There is a voltage drop across each of the three inter-patch spaces within the body and this is used to calculate the location of the monitored electrode(s) in three-dimensional space. The LocaLisa system can track up plural electrodes simultaneously. The electrode locations are shown on a three axis coordinate grid on the monitor. One limitation of the LocaLisa system is that the system achieves the best accuracy when the electric field gradients are uniform. Distortions to these electric fields cause inaccuracies in the rendered position of the electrodes. Surgical procedures produce air voids within the thoracic cavity that can cause electric field distortions. The electrodes that are being tracked must also maintain contact with conductive tissue at all times to maintain their position in the coordinate system. These issues make electric field based navigation challenging for surgical applications.

Another system is referred to as the NavX system. The NavX system utilizes electric field based navigation which is similar to that used by the above-described LocaLisa system. Electric fields passing in three axes through the body are used to track electrodes within the cardiovascular system. The NavX system can provide navigation, image integration, and electrical activation mapping using a non-contact mapping balloon catheter placed within the region to be mapped. This system can potentially take a previously acquired CT or MR scan and register the scan to the patient for real-time electrode positioning so that the computer can show the catheters in a real image of the heart of the patient. The NavX system has the same shortcomings as the LocaLisa system if used for medical procedures in the thoracic cavity.

Another system is referred to as the Carto system. The Carto system uses electromagnetic field based tracking technology. Three small coils are mounted on a catheter and an antenna pad is placed under the patient that can allow the system to sense the three-dimensional location of these coils and display the location on a computer. The user can move the catheter around and map the geometery of the region of interest and display the region on the computer. The Carto system can also perform electroanatomic activation mapping of the endocardial surfaces of the region which the catheter is in. This is done by moving the catheter around and taking data at a number of points of interest within the region. The accuracy of the chamber rendering is only as good as the number of points collected. The accuracy of the Carto system is generally not sensitive to air voids within the body created during medical procedures. One drawback of the Carto system is that the Carto system requires three coils oriented in three different axes to be collocated on the device or instrument to be tracked. Moreover, only one coil loaded device can be tracked at the same time. This can be challenging for devices with limited available space.

Another system is referred to as the Real-Time Positioning (RPN) system. The RPN system is an ultrasound based system that incorporates ultrasound transducers mounted in the catheters to be tracked. These transducers emit ultrasonic energy that is received by transducers on other catheters within the cardiovascular system. The RPN system then displays the relative positions of all of the transducers and renders images of the catheters that the transducers are mounted on. The RPN system is sensitive to air voids or differences in the speed of sound within various types of tissue. These issues make use of the RPN system challenging for surgical applications.

Another system is referred to as the FluoroNav system. The FluoroNav system utilizes an electromagnetic field transmitter that can be attached to the image intensifier of a fluoroscope used in spinal surgery. It transmits three alternating magnetic fields that can be received by coils within the region of interest. This transmitter also contains a matrix of small metal spheres that are used to normalize the fluoroscopic image. Fluoroscopic images are acquired in one or more directional orientations. These images of the spinal anatomy are then viewed by a surgeon who is able to track medical instruments within the field of interest. Each medical instrument has at least one receiving coil that allows the instrument to which the coil is attached to be tracked in three-dimensional space with respect to the previously acquired fluoroscopic image. This allows a surgeon to manipulate medical instruments with minimal x-ray exposure.

The above-described navigation systems are generally not suitable for precise guidance of cardiothoracic medical instruments due to the issues with respiration and cardiac motion, for example, which limit accurate placement.

### SUMMARY

The present invention accordingly provides medical instruments and methods to positionally monitor, guide, track, or otherwise navigate at least a portion of one or more medical instrument positioned inside a predetermined portion of the anatomy of a patient during a medical procedure being performed on the patient. Exemplary medical procedures include pre-operative procedures, surgical procedures, and outpatient procedures. More particularly, exemplary embodiments of the present invention provide devices, instruments, systems, and methods useful to navigate one or more medical instrument in three-dimensional space and real-time during a medical procedure. Advantageously, a visual representation of a medical instrument can be viewed together with previously acquired imaging of the predetermined portion of the anatomy of the patient during the medical procedure.

Exemplary embodiments of the present invention thus provide navigation systems comprising one or more medical instruments having one or more electromagnetically detectable receiver coils integrated with at least a portion of a medical instrument and an electromagnetic transmitter configured to identify information related to one or both the position and shape (current or changing) of a medical instrument. More particularly, an electromagnetic coil functions, alone or in combination with at least one additional electromagnetic coil, to provide the position of at least a portion of a medical instrument in three-dimensional space and in real-time during a medical procedure being performed on a patient. That is, an electromagnetically detectable receiver coil functions like an antenna.

In an exemplary embodiment of the present invention, the shape (current or changing) of at least a portion of a medical instrument having at least one electromagnetic coil integrated with the medical instrument can also be provided. In use, one or both of the position and shape (current or changing) of at least a portion of a medical instrument can be viewed on a display device, such as a monitor of a computer system or the like, during a medical procedure being performed on a patient.

In exemplary embodiments of the present invention, previously acquired imaging of a predetermined portion of the anatomy of a patient is used together with medical instruments in accordance with the present invention. Previously acquired imaging may comprise one or more of fluoroscopic imaging, ultrasonic imaging, computed tomographic (CT) imaging, and magnetic resonance (MR) imaging, for example. In particular, the predetermined portion of the anatomy of the patient typically comprises the portion of the anatomy of the patient on which a medical procedure is being performed. During the procedure, one or both of the position and shape (current or changing) of at least a portion of a medical instrument is displayed on a display device.

Additionally, the previously acquired imaging is preferably registered with and simultaneously displayed with one or both of the position and shape (current or changing) of at least a portion of a medical instrument during the medical procedure. The present invention thus enables a surgeon to manipulate one or more medical instrument within a predetermined portion of the anatomy of a patient during a medical procedure while simultaneously viewing, in three-dimensional space and in real-time, the position of a medical instrument within a computer rendered image of the actual anatomy of the patient.

In exemplary embodiments of the present invention, information from a medical instrument and the previously acquired imaging is provided to a user interface. The user interface preferably functions to process spatial information related to a medical instrument and the previously acquired imaging so the spatial information and imaging can be registered with each other and provided on a display device. The user interface provides the surgeon with the real-time video image in conjunction with a rendering of the anatomy that includes a depiction of the orientation and position of the surgical instrument in relation to specific anatomical sites. This way, the surgeon will know what portion of the anatomy is being viewed endoscopically and what portion of the anatomy the surgical instrument is in proximity to. This will aid the surgeon in situations where the limited space and close proximity to the tissue make endoscopic video of limited value in determining the identity of the anatomical structures being viewed.

In an exemplary application, the present invention can be advantageously used for minimally invasive cardiothoracic medical procedures. The present invention facilitates electromagnetic navigation of one or more medical instrument and advantageously provides information related to the proximity a medical instrument to structures such as cardiac chambers, great vessels, and nerves, for example. The present invention also allows movement of a medical instrument safely within the thoracic cavity of a patient during a medical procedure without direct or endoscopic visualization. Additionally, the present invention provides the ability to guide the delivery of one or more therapeutic devices to precise predetermined and targeted locations within the thoracic cavity. Moreover, the present invention provides the ability to guide placement of one or more therapeutic electrodes, leads, or the like to precise predetermined and targeted locations within the thoracic cavity for targeted therapy delivery.

In another aspect of the present invention a method of monitoring the position of a medical instrument in three-dimensional space and in real-time during a medical procedure being performed on a patient is provided. The method comprises: acquiring imaging of a predetermined portion of the anatomy of a patient; providing a medical instrument comprising one or more electromagnetically detectable receiver coil, the position of which can be determined in three-dimensional space and in real-time; placing the at least a portion of the medical instrument within the predetermined portion of the anatomy of the patient; identifying the position, in three-dimensional space and in real-time, of the at least a portion of the medical instrument placed within the predetermined portion of the anatomy of the patient; displaying the previously acquired imaging of the predetermined portion of the anatomy of the patient on a display screen; and indicating the position of the at least a portion of the medical instrument on the display screen.

In another aspect of the present invention a medical instrument is provided. The medical instrument is preferably configured so the position of at least a portion of the medical instrument can be monitored in three-dimensional space and in real-time during a medical procedure being performed on a patient. The medical instrument preferably comprises a body portion; and an electromagnetically detectable receiver coil integrated with the body portion and configured so the position of at least a portion of the one or more electromagnetically detectable receiver coil can be determined in real-time and in three-dimensional space.

In another aspect of the present invention a medical navigation system is provided. The medical navigation system can monitor the position of a medical instrument in three-dimensional space and in real-time during a medical procedure being performed on a patient. The medical navigation system preferably comprises at least one medical instrument comprising a body portion comprising an electromagnetically detectable receiver coil integrated with the body portion and configured so the position of at least a portion of the electromagnetically detectable receiver coil can be determined in three-dimensional space and in real-time; an electromagnetic transmitter configured to transmit a signal to the at least a portion of the electromagnetically detectable receiver coil; a source of previously acquired imaging of a predetermined portion of the anatomy of the patient; and a display screen configured to display the previously acquired imaging of a predetermined portion of the anatomy of the patient; a user interface configured to receive information from the electromagnetic transmitter and display the previously acquired imaging of the predetermined portion of the anatomy of the patient together with a real-time graphical and positional representation of at least a portion of the medical instrument.

As noted above, exemplary aspects of the present invention provide methods of electromagnetically monitoring, guiding, tracking, or otherwise navigating placement of one or more medical instruments, devices, or both within a predetermined portion of the anatomy of a patient during a medical procedure being performed on the patient. Exemplary medical instruments that can be used in accordance with the present invention comprise, for example, endoscopic instruments, endoscopic visual imaging instruments, such as those comprising one or both of a light guide and camera as well as those comprising one or both of a rigid proximal section and a flexible distal section, tissue ablation instruments, such as those comprising one or more electrodes, tissue ligation instruments, dissection instruments, rigid medical instruments, and flexible medical instruments. Additionally, an instrument comprising one or both of an endoscopic light guide and camera having a sheath or removable cover, wherein one or more electromagnetically detectable receiver coil is integrated with the sheath or removable cover can be used in accordance with the present invention.

Exemplary medical devices that can be used in accordance with the present invention include fiducial marking devices, esophageal devices, transesophageal ultrasound imaging devices or transducers, transthoracic ultrasound imaging devices or transducers, transvenous or intracardiac ultrasound imaging devices, catheters, or transducers, flexible surgical guiding devices (can be used to determine shape or changes in shape of a medical instrument), and tracheal devices, for example. Additionally, devices comprising a catheter-like insert having one or more electromagnetically detectable receiver coil that can be passed through the lumen of a larger catheter to track the path of the lumen of the larger catheter can be used in accordance with the present invention.

In another exemplary aspect of the present invention, a method of cataloging, in three-dimensional space, the location of one or more ablated cardiac or other tissue sites where ablation has been performed is provided. Cataloging of sites can be performed by the surgeon by marking the appropriate anatomical sites with points or markers with embedded notations on the anatomical rendering. For example, the surgeon can use an input device (such as a foot pedal) at the appropriate time to place a marker on the anatomical rendering. By marking such ablated sites, additional placements of ablation tools may be properly facilitated to ensure intersection of new ablation lines with previously ablated regions or lines of tissue.

In another exemplary aspect of the present invention, a method of coupling a visual real-time camera image of a predetermined portion of the anatomy of a patient from an endoscopic instrument with the electromagnetically provided position of such instrument within a predetermined portion of the anatomy of the patient is provided. Such camera image and position information is preferably provided on a display device. Additionally, such camera image and positional information can also be provided with previously acquired imaging of the predetermined portion of the anatomy of the patient on the display screen.

As an example, a rendered cartoon image of the camera and associated surgical instrument is displayed on the screen in the actual correct orientation in space with respect to the rendered anatomical image. Along side of this depiction of the camera associated with a surgical instrument is the actual endoscopic video of the anatomy. Such side by side display provides confidence to the surgeon regarding the identity of the targeted anatomy being approached. When registering a previously acquired anatomical image to the real-time anatomy, the camera monitored instrument is first used to approach a series of fiducial points, all easily identified on the preacquired image as well as on the real-time anatomy. By touching the instrument on such points and assigning each point to an easily identifiable point on the preacquired image, the image can then be registered to the real-time anatomy.

In another exemplary aspect of the present invention a method of providing a visual real-time endoscopic image of a predetermined portion of the anatomy of a patient simultaneously with previously acquired imaging of the predetermined portion of the anatomy of the patient is provided. Preferably, the directional vector of the image is aligned with the normal vector of the viewing direction of the previously acquired imaging of the predetermined portion of the anatomy of the patient. A preferred method of orienting the anatomical rendering and cartoon image of the camera or associated surgical instrument comprises adjusting the anatomical rendering at all times to maintain the cartoon rendering of the camera or associated surgical instrument in a normal or perpendicular orientation to the anatomy in the field of view. The rendered anatomy reorients to always provide immediate feedback to the surgeon regarding the anatomy being visualized with the camera.

In another exemplary aspect of the present invention a method of image registration is provided. Preferably, one or more fiduciary marking devices are used to register previously acquired imaging of a predetermined portion of the anatomy of a patient with the actual anatomy of the patient. In an exemplary embodiment, a fiduciary marking device may comprise one or more of a fixed surface fiduciary marking device and an indwelling fiduciary marking device, or combinations thereof. Preferably, fiduciary marking devices are detectable by one or more non-invasive imaging techniques such as an x-ray fluoroscopy, computed tomography, magnetic resonance, and ultrasound imaging, and preferably comprises one or more electromagnetically detectable receiver coil facilitating identification of the location such fiduciary marking devices, in three-dimensional space, and used as a reference for real-time registration.

Advantageously, fiduciary marking devices can be used to monitor real-time positional changes of predetermined anatomical structures. Fiduciary marking devices can also be used to monitor real-time changes such as respiration, cardiac motion, and intestinal peristalsis. Fiduciary marking devices may further include devices positioned in one or both of the esophagus and trachea. A fiduciary marking device may be registered one or more times or monitored in real-time to update and correct such registration as needed.

In another exemplary aspect of the present invention, a fiduciary marking device can be fixed in location on or within the anatomy of a patient by using one or more of an adhesive, a tissue fixation screw or helix, suction, an inflatable balloon, expandable structures, and physical pressure.

### BRIEF DESCRIPTION OF THE DRAWINGS

The accompanying drawings, which are incorporated in and constitute a part of this disclosure, illustrate several aspects of the present invention and together with description of the exemplary embodiments serve to explain the principles of the present invention. A brief description of the drawings is as follows:

FIG. 1 is a perspective view of an exemplary medical instrument comprising an electromagnetically detectable receiver coil and in particular, showing an exemplary medical instrument comprising plural electromagnetically detectable receiver coils in accordance with the present invention.

FIG. 2 is a perspective view of the distal end of the medical instrument shown in FIG. 1.

FIG. 3 is a schematic perspective view of an exemplary medical instrument comprising an electromagnetically detectable receiver coil and in particular, showing an exemplary medical instrument comprising plural electromagnetically detectable receiver coils positioned relative to an outside surface of a shaft portion of the medical instrument, in accordance with the present invention.

FIG. 4 is a schematic perspective view of another exemplary medical instrument comprising an electromagnetically detectable receiver coil and in particular, showing an exemplary medical instrument comprising plural electromagnetically detectable receiver coils positioned relative to an internal core of the medical instrument, in accordance with the present invention.

FIG. 5 is a schematic perspective view of an exemplary lead that can be used in accordance with the present invention.

FIG. 6 is a schematic perspective view of an exemplary medical instrument that can be used for implanting the lead shown in FIG. 5 in accordance with the present invention.

FIG. 7 is a schematic perspective view of an exemplary medical instrument assembly including the lead of FIG. 5 positioned in the medical instrument of FIG. 6 in accordance with the present invention.

FIG. 8 is a schematic perspective view of an exemplary medical instrument in accordance with the present invention comprising the lead and medical instrument assembly shown in FIG. 7 as positioned within the delivery sheath shown in FIG. 3 in accordance with the present invention.

### DETAILED DESCRIPTION

The exemplary embodiments of the present invention described herein are not intended to be exhaustive or to limit the present invention to the precise forms disclosed in the following detailed description. Rather the exemplary embodiments described herein are chosen and described so those skilled in the art of cardiothoracic surgery, in particular, can appreciate and understand the principles and practices of the present invention.

In preferred embodiments, the present invention provides systems and methods that can advantageously display previously acquired images of a predetermined portion of the anatomy of the thoracic cavity (or other anatomy of interest) of a patient while additionally displaying one or more image and precise spatial information related to one or more medical instruments that are inserted into the thoracic cavity, such as during cardiothoracic surgery, for example. Such medical instruments, for example, may be hand held, remotely controlled by magnetic fields, or robotically held or manipulated. Other medical instruments, known or future developed, can benefit from the systems and methods of the present invention.

Each medical instrument that is tracked in real-time includes at least one electromagnetically detectable receiver coil integrated with the medical instrument in accordance with the present invention. An electromagnetically detectable receiver coil facilitates the ability to track a medical instrument and to have an image of the medical instrument rendered on a display screen, such as a display screen associated with a computer device.

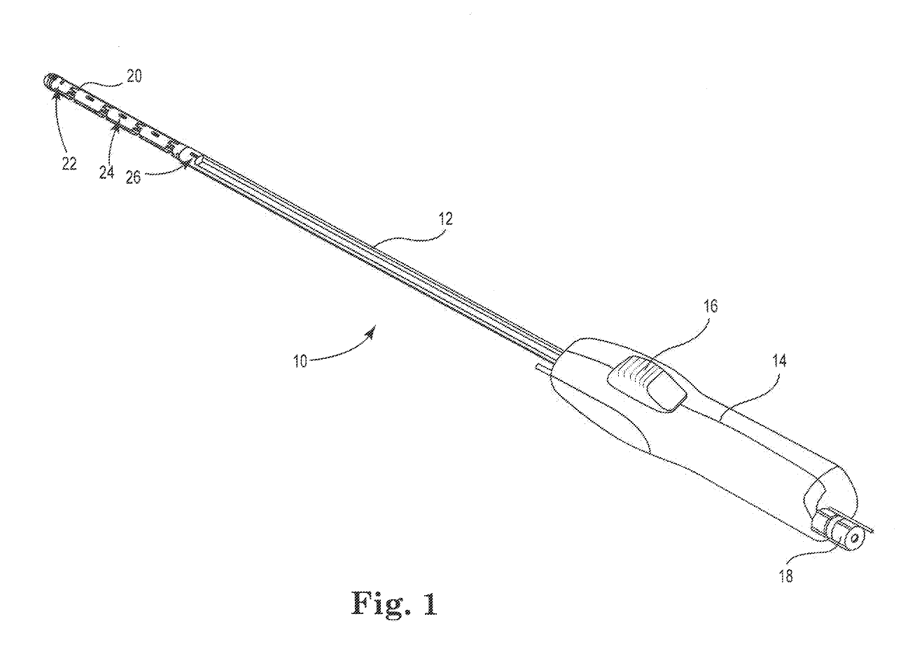

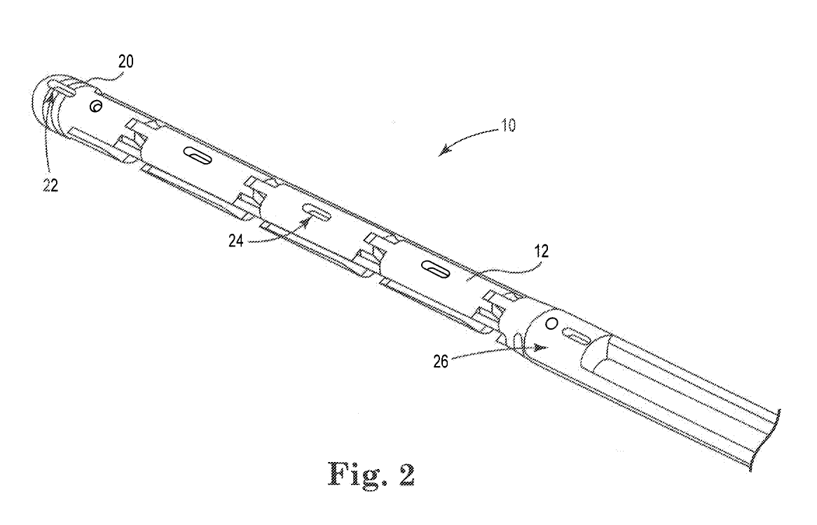

An exemplary medical instrument 10 in accordance with the present invention is illustrated in FIGS. 1 and 2 and comprises an ablation instrument for purposes of illustration. Medical instrument 10 comprises shaft 12, handle 14 having switch 16, and power cord 18. Referring to FIG. 2 in particular, distal end 20 of medical instrument 10 is illustrated in greater detail. As shown in exemplary medical instrument 10, first, second, and third electromagnetically detectable receiver coils 30, 22, 24, and 26, respectively, are provided at distal end 20 of medical instrument 10. Any desired number of electromagnetically detectable receiver coils can be used in accordance with the present invention.

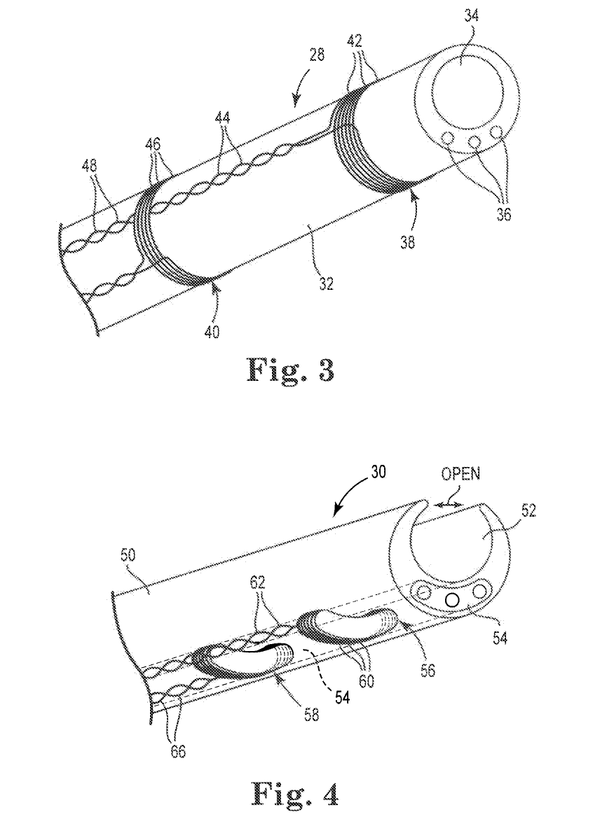

FIGS. 3 and 4, illustrate medical instruments, 28 and 30, respectively, in accordance with the present invention. Medical instruments, 28 and 30, as illustrated, comprise endoscopic instruments. Referring to FIG. 3 initially, medical instrument 28 comprises shaft 32, working lumen 34, fiber-optic (one or both of light and vision) and irrigation ports generally referred to with reference numeral 36, and first and second electromagnetically detectable receiver coils, 38 and 40, respectively. First electromagnetically detectable receiver coil 38 comprises plural windings 42 and signal leads 44. Similarly, second electromagnetically detectable receiver coil 40 comprises plural windings 46 and signal leads 48.

Referring now to FIG. 4, medical instrument 30 comprises shaft 50, open lumen 52, and fiber-optic and irrigation bundle core 54. As illustrated, medical instrument 30 comprises first and second electromagnetically detectable receiver coils, 56 and 58, respectively. First and second electromagnetically detectable receiver coils, 56 and 58, respectively, are operatively wrapped around core 54. First electromagnetically detectable receiver coil 56 comprises plural windings 60 and signal leads 62. Similarly, second electromagnetically detectable receiver coil 58 comprises plural windings 64 and signal leads 66.

Integration of one or more navigation coil with a medical instrument in accordance with the present invention can be achieved in any desired manner including techniques known or future developed. For example, a navigation coil can be structurally integrated with a desired medical instrument. A navigation coil can also be attached or otherwise provided on the surface of a desired medical instrument.

In an exemplary embodiment, for example, one or more navigation coil can also be provided on an outside surface of a sheath. A removable sheath would preferably comprise a disposable structure that would allow insertion of an endoscopic instrument within the sheath. The sheath would serve to protect the endoscopic instrument from body fluids, for example, and the sheath could advantageously provide one or more working lumen to allow one or more of suction, irrigation, and passage of guide-wires, catheters or similar flexible, or polymeric devices or instruments through the sheath and into the working region at the distal end of the endoscopic instrument. Tracking coils are preferably integrated with the sheath so the position of one or more of the distal end, the flexible portion, and the rigid portion of the endoscopic instrument can be tracked and displayed in three-dimensional space in accordance with the present invention.

Electromagnetic navigation in accordance with the present invention, in a preferred exemplary embodiment, utilizes a system that preferably transmits three separate electromagnetic fields that are received or otherwise sensed by one or more electromagnetically detectable receiver coils integrated with the medical instrument to be tracked. Preferably, at least one coil is used to monitor the three-dimensional location of that coil in three-dimensional space as well as the medical instrument the coil is integrated with. Use of additional coils advantageously adds definition to the shape (current or changing) and path of certain flexible medical instruments such as those that include flexible or malleable instruments. Accurate registration of previously acquired anatomical images can be performed using one or more of surface fiducial registration points, internal, implanted, and indwelling reference devices, for example. The form of reference points required to register the image to the true anatomy, depends on the accuracy needed for the particular procedure and anatomy of interest.

Medical instruments in accordance with the present invention are preferably designed to optimize the inductance signal and minimize interference between an electromagnetically detectable receiver coil and a transmitted electromagnetic field. Accordingly, medical instruments in accordance with the present invention may comprise any material suitable for use as a medical instrument such as stainless steel, titanium, and polymers, for example. Exemplary polymers include liquid crystal polymers, polysulfone, polythenylene sulfide, polyetheretherketone, and polyetherimide, for example.

In an exemplary embodiment, initial imaging of the thoracic cavity (or other anatomy of interest) of a patient includes using one or more of: 1) fluoroscopy, 2) computed tomography (CT), 3) magnetic resonance (MR) imaging, and 4) two-dimensional or three-dimensional ultrasound imaging prior to a medical procedure. In an exemplary embodiment, the present invention can use technology related to that of the Medtronic FluoroNav™ system.

The initial imaging, referred to as previously acquired imaging throughout this document, is preferably carried out by first placing fiduciary marking devices on specific points on or in the body of the patient. Such fiduciary marking devices may include marking devices that can be easily identified on the images by use of the appropriate contrast materials sensitive to the particular imaging technique to be used. These markers can be attached to the skin, implanted, subcutaneous, placed in the trachea, bronchi, or esophagus, or inserted into the cardiovascular system, for example.

One exemplary marker comprises a fiduciary catheter-like device having plural electromagnetically detectable receiver coils that can be placed via the venous system through the caval veins (inferior and/or superior vena cava) and extended into various additional portions of the right side of the heart including one or more of the right atrial appendage, the coronary sinus, the right ventricle, the inter-ventricular septum, the right ventricular apex, the right ventricular outflow tract, and the pulmonary arteries. Delivery to sites such as the pulomonary arteries could be aided by the addition of a balloon on the end of the fiduciary catheter to make use of blood flow to urge the balloon downstream into the distal end of the right side of the cardiovascular system and into one or more of the pulmonary arteries.

Additionally, such a fiduciary marking catheter-like device could also be placed in the arterial side of the cardiovascular system whereby the device would be introduced via an artery into the ascending aorta and extended through the descending aorta (or into superior arterial vessels) and into one or more of the aortic valve, into the left ventricle, the inter-ventricular septum, the left ventricular apex, the mitral valve annulus, the left atrium, the left atrial appendage, and the pulmonary veins.

An advantage of positioning fiduciary marking devices in one or more of the esophagus and trachea would be that such devices can track respiration effects in real-time on the posterior aspects of the heart. The electromagnetically detectable receiver coils or fiducial marking devices can be integrated with tracheal tubes used for patients on a respirator. An esophageal reference, in particular, would provide precise information of the location of the esophagus during procedures involving ablation of regions of the left atrium, for example.

Additionally, fiducial marking devices, reference devices, and catheters can be placed in and around one or more of the heart and pericardial space to define the real-time precise location of such surfaces and structures. With one or more of a fiduciary catheter or catheters and markers in place, imaging can be performed with these fiduciary marking devices in place at various desired locations. Imaging is preferably performed with regard to respiration and cardiac cycle of the patient, for example, so these motions can advantageously be accounted for during the timing of the acquisition of the images.

A surgeon or physician preferably determines placement of such fiduciary marking devices considering the predetermined portion of the anatomy of the patient and where the highest accuracy of positional information of medical instruments with respect to the anatomical structures is desired. Advantageously, placement of catheter-like fiduciary marking devices can be performed using minimal fluoroscopy or other suitable minimally invasive imaging technique.

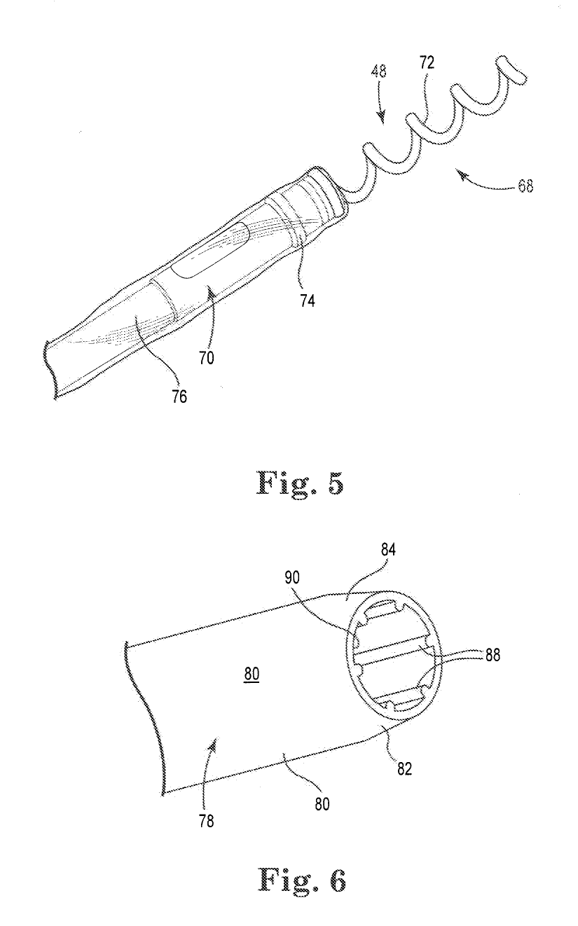

An exemplary application of the present invention relates to implantation of one or more epi lead. With reference to FIGS. 5-8 and FIG. 5 initially an exemplary epi lead 68 is illustrated. As shown, lead 68 comprises body 70 connected to helix 72 by crimped connector 74. At least a portion of body 70 and crimped connector 74 are positioned within sleeve 76. Preferably, sleeve 76 comprises a urethane tube, however, other similarly functioning materials can be used in accordance with the present invention.

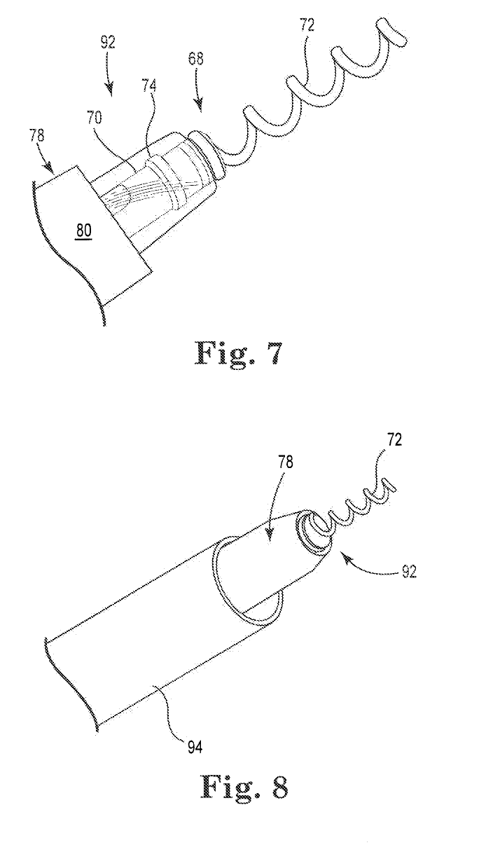

Referring next to FIG. 6, insertion tool 78 is illustrated and can be used for implanting lead 68 at a predetermined portion of the anatomy of a patient during a medical procedure, such as a surgical procedure, for example. As shown, insertion tool 78 comprises body 80 having chamfer 82 at distal end 84 of body 80. Insertion tool 78 further comprises internal, generally cylindrical, region 86. Internal region 86 comprises plural internal splines 88 each of which extend from inside surface 90 of region 86. Internal splines 88 function to engage sleeve 76 of lead 60 when lead 60 is positioned in internal region 86 of insertion tool 78 to provide assembly 92 as can be seen in FIGS. 7 and 8. Insertion tool 78 can be made from any desired material with an exemplary preferred material comprising a thermoplastic polyester elastomer such as Hytrel® 8238, for example.

In FIG. 8, assembly 92 is shown positioned in the working lumen of endoscope 94 in accordance with the present invention. Endoscope 94 may comprise an endoscope such as is shown in FIG. 3, for example. A preferred endoscope comprises a PS Medical Channel™ Neuroendoscope having model number 2232-003. One or more electromagnetically detectable receiver coil, in accordance with the present invention, may be integrated with any of lead 68, insertion tool 78, and endoscope 94.

Another exemplary application of the present invention relates to pulmonic valve replacement using a transvascular approach. This particular procedure preferably uses preliminary imaging with one or more of skin surface fiduciary markers and a fiduciary marking catheter placed through the venous system into the right ventricle outflow tract and to the site of the pulmonic valve annulus in accordance with the present invention.

After preliminary imaging is complete and the patient is in the operating room, the preliminary imaging is preferably registered to the patient using one or more of the surface fiduciary markers and the internal catheter to provide high accuracy registration between the imaging and the actual anatomy of the patient in the region of interest at the pulmonic valve annulus. As the procedure continues, the fiduciary catheter is preferably removed and the valve delivery catheter is then preferably advanced into the site of the pulmonic valve for deployment. At any desired stage of the procedure, a surgeon or physician can advantageously use the image guidance navigation system in accordance with the present invention to view the real-time location and advancement of the valve delivery catheter. Moreover, the surgeon or physician can advantageously view the motion of the catheter through the cardiovascular system all the way to the site of deployment at the pulmonic valve annulus using the image guidance navigation system in accordance with the present invention. Using the image guided navigation system in accordance with the present invention can reduce or eliminate the need for fluoroscopy during the delivery process, which would advantageously benefit both patient and medical personnel.

Another exemplary application of the present invention relates to minimally invasive epicardial ablation to treat atrial fibrillation. This procedure benefits from the ability to dissect a path around the cardiac anatomy through which the ablation instrument can be placed to create the appropriate lesions from the epicardial aspect. Assuming the procedure in this case would be performed from the right side of the patient, the structures of interest to the surgeon upon port entry into the thoracic cavity would be the location of the pericardial sac and associated structures such as the phrenic nerve. Also of interest would be the location and courses of the caval veins, pulmonary arteries, and pulmonary veins. The location of the lung surface is also of interest and the location of the lung surface can be tracked by placement of a device containing one or more tracking coil on the surface of the lung in accordance with the present invention. Also of interest during ablation procedures is the relative location of the esophagus with respect to the location of one or more medical instruments such as an ablation tool.

Regarding epicardial ablation, for example, it may also be advantageous to use an endoscopic camera or light guide to allow visual imaging of a surgical site. Such camera instrument can produce an image that can be displayed on a display screen independently or such visual camera image can be integrated with the display from the navigation system. That is, the camera instrument could have one or more electromagnetically detectable receiver coil integrated with the camera instrument in accordance with the present invention so the camera instrument can be tracked in three-dimensional space by the navigation system. The location in three-dimensional space of the camera instrument can advantageously be integrated to display a visual image in real-time so the surgeon would know what anatomical structures are being viewed in the visual camera image.

Endoscopic camera viewing instruments can be equipped with one or more electromagnetically detectable receiver coil in accordance with the present invention. Use of one or more electromagnetically detectable receiver coil can advantageously define the location of any desired portion of the instrument such as the proximal and distal portions of the instrument as desired or preferred for use with a particular procedure. In the case of flexible or deflectable endoscopic tools, more than one coil can be used to define the location and path of both proximal and flexible distal portions of such instruments. Moreover, a flexible surgical guiding device can be used to identify the shape as well as a change in shape of a predetermined portion of a medical instrument.

The need for tracking and navigation of endoscopic visual instruments is increased when the space such instruments have to operate in is small. This lack of space creates a situation where because the surgeon sees such a small field of view, it is difficult to accurately identify the relevant location within the anatomy of the patient. This makes it particularly difficult for the surgeon to accurately perform a desired procedure. For example, procedures directed to dissection and separation of layers of tissue can be challenging to perform. These dissections are important to minimally invasive procedures where instruments need to work in small spaces and sometimes only virtual spaces between structures.

An advantage of systems and methods in accordance with the present invention is the ability to work in small or virtual spaces. Moreover, an advantage of systems and methods in accordance with the present invention is that one or both lungs would typically not need to be deflated or would not require the extent of deflation required for purely endoscopic procedures.

In terms of the atrial fibrillation treatment procedure, the precise location of the caval veins and other structures can be registered to previously acquired imaging using fiducial marking catheters placed in the venous cardiovascular system as previously described. One challenging dissection procedure involves separation of the pericardial reflections that are located between the superior pulmonary veins. In this region, the surgeon works carefully around the atrial walls, pulmonary veins, and in particular, the pulmonary arteries. Placing a fiduciary marking device into one or more of the pulmonary arteries advantageously helps to provide precise registration of these structures of the patient early in the procedure in the operating room. Such precise location registration advantageously aids the surgeon in performance of the dissections of these pericardial reflections.

Another exemplary application of the present invention relates to the delivery and placement of a stent-graft to repair abdominal aortic or thoracic aortic aneurisms. In this procedure, acquisition of a detailed CT or MR image of the aortic arterial system is desired, not only to show the aneurism in detail, but also to identify the branch sites of numerous arteries. These branch arteries of interest include the carotid, brachiocephalic trunk, subclavian, bronchial, phrenic, hepatic, cephalic trunk, splenic, mesenteric, renal, lumbar, and iliac arteries. It is useful to identify the branch locations of these arteries when placing stent-grafts in the aorta that may occlude such arteries.

For the stent-graft delivery procedure, the delivery catheter is preferably equipped with one or more navigation coils in accordance with the present invention that allow precise tracking of the delivery system through the aortic anatomy. The previously acquired imaging is useful in determining the optimal graft placement site that would prevent further distension and rupture. Branch artery locations are preferably avoided where possible but when the stent-graft is placed in a location that occluded such a vessel, the previously acquired imaging can help to guide the placement of a peroration and side branch perfusion channel to supply the occluded artery through the wall of the stent-graft.

The present invention has now been described with reference to several exemplary embodiments thereof. The entire disclosure of any patent or patent application identified herein is hereby incorporated by reference for all purposes. The foregoing disclosure has been provided for clarity of understanding by those skilled in the art cardiothoracic surgery, in particular. No unnecessary limitations should be taken from the foregoing disclosure. It will be apparent to those skilled in the art of cardio thoracic surgery, in particular, changes can be made in the exemplary embodiments described herein without departing from the scope of the present invention. Thus, the scope of the present invention should not be limited to the exemplary structures and methods described herein, but only by the structures and methods described by the language of the claims and the equivalents of those claimed structures and methods.

What is claimed is:

1. A method of monitoring the position of a medical instrument in three-dimensional space and in real-time during a medical procedure being performed on a patient, the method comprising:
acquiring imaging of a predetermined portion of the anatomy of a patient;
providing a medical instrument, the medical instrument comprising one or more electromagnetically detectable receiver coils, the position of which can be determined in three-dimensional space and in real-time;
placing the at least a portion of the medical instrument within the predetermined portion of the anatomy of the patient;
identifying the position, in three-dimensional space and in real-time, of the at least a portion of the medical instrument placed within the predetermined portion of the anatomy of the patient;
displaying the previously acquired imaging of the predetermined portion of the anatomy of the patient on a display screen; and
indicating the position of the at least a portion of the medical instrument on the display screen.

2. The method of claim 1, comprising acquiring imaging of the predetermined portion of the anatomy of the patient using one or more of x-ray fluoroscopy, computed tomography, magnetic resonance, and ultrasound imaging.

3. The method of claim 1, comprising acquiring imaging of the predetermined portion of the anatomy of the patient prior to the medical procedure being performed on the patient.

4. The method of claim 1, comprising displaying an image of at least a portion of the predetermined portion of the anatomy of the patient acquired from an endoscopic instrument together with the previously acquired imaging and the indication of the position of the at least a portion of the medical instrument on the display screen.

5. The method of claim 4, wherein the image acquired from the endoscopic instrument comprises a real-time image.

6. The method of claim 1, comprising using information comprising the position, in 3-dimensional space, of at least one fiducial marking device positioned within the predetermined portion of the anatomy of the patient to register the previously acquired imaging of the predetermined portion of the anatomy of the patient with the actual anatomy of the patient.

7. The method of claim 1, comprising transmitting an electromagnetic signal to the one or more electromagnetically detectable receiver coil.

8. The method of claim 1, wherein the medical procedure comprises cardiothoracic surgery.

9. A medical instrument configured so the position of at least a portion of the medical instrument can be monitored in three-dimensional space and in real-time during a medical procedure being performed on a patient, the medical instrument comprising:
a body portion; and
an electromagnetically detectable receiver coil integrated with the body portion and configured so the position of at least a portion of the one or more electromagnetically detectable receiver coils can be determined in real-time and in three-dimensional space.

10. The medical instrument of claim 9, comprising plural electromagnetically detectable receiver coils.

11. The medical instrument of claim 9, comprising a generally cylindrical shaft wherein the electromagnetically detectable receiver coil is wound around an outside surface of the generally cylindrical shaft.

12. The medical instrument of claim 9 comprising a generally cylindrical shaft having a removable core wherein the electromagnetically detectable receiver coil is wound around an outside surface of the removable core.

14. The medical instrument of claim 9, wherein the medical instrument comprises an endoscopic tool having a removable sheath surrounding a portion of the endoscopic tool.

15. The medical instrument of claim 9 in combination with an electromagnetic transmitter configured to transmit a signal to the at least a portion of the electromagnetically detectable receiver coil.

16. A medical navigation system that can monitor the position of a medical instrument in three-dimensional space and in real-time during a medical procedure being performed on a patient, the medical navigation system comprising:
at least one medical instrument comprising a body portion comprising an electromagnetically detectable receiver coil integrated with the body portion and configured so the position of at least a portion of the electromagnetically detectable receiver coil can be determined in three-dimensional space and in real-time;
an electromagnetic transmitter configured to transmit a signal to the at least a portion of the electromagnetically detectable receiver coil;
a source of previously acquired imaging of a predetermined portion of the anatomy of the patient; and
a display screen configured to display the previously acquired imaging of the predetermined portion of the anatomy of the patient;
a user interface configured to receive information from the electromagnetic transmitter and display the previously acquired imaging of the predetermined portion of the anatomy of the patient together with a real-time graphical and positional representation of at least a portion of the medical instrument.

17. The system of claim 16, wherein the body portion of the medical instrument comprises plural electromagnetically detectable receiver coils.

18. The system of claim 16, comprising at least one fiducial marking device that can be positioned within the predetermined portion of the anatomy of the patient to register the previously acquired imaging of the predetermined portion of the anatomy of the patient with the actual anatomy of the patient.

19. The system of claim 16, comprising an endoscopic viewing instrument distinct from the medical instrument.

20. The system of claim 16, configured for use in cardiothoracic surgery.
